(19) United States
(12) Patent Application Publication
(10) Pub. No.: US 2016/0206776 A1
(43) Pub. Date: Jul. 21, 2016

(54) APPARATUS FOR PRODUCING A DOUGHY BONE CEMENT SHAPE

(71) Applicant: BIOMET MANUFACTURING, LLC, Warsaw, IN (US)

(72) Inventors: John M. McDaniel, Warsaw, IN (US); Daniel B. Smith, Warsaw, IN (US)

(21) Appl. No.: 14/914,085

(22) PCT Filed: Aug. 27, 2014
(86) PCT No.: PCT/US2014/052867

§ 371 Date: Feb. 24, 2016

Related U.S. Application Data
(60) Provisional application No. 61/870,366, filed on Aug. 27, 2013.

(51) Int. Cl. A61L 24/06; A61L 24/04; A61B 17/88

(52) U.S. Cl. CPC A61L 24/06; A61B 17/8833; A61L 24/046; A61L 2430/02

(57) ABSTRACT

A bone cement forming kit, comprising a malleable bone cement material, and a flexible membrane encapsulating the malleable bone cement material, wherein the flexible membrane has at least one removable panel and is configured to form an aperture that exposes the malleable bone cement material once the panel is removed.

### CROSS-REFERENCE TO RELATED APPLICATIONS

This application is related to and claims priority to U.S. Provisional Patent Application Ser. No. 61/870,366, which was filed on Aug. 27, 2013, the complete and entire disclosure of which is hereby expressly incorporated by reference herein.

### TECHNICAL FIELD

The present teachings relate to doughy bone cement materials, kits comprising doughy bone cement materials and associated methods for producing doughy bone cement shapes.

### BACKGROUND OF THE DISCLOSURE

The statements in this section merely provide background information related to the present disclosure and should not be construed as constituting prior art.

The natural joints of the human body often undergo degenerative changes due to various etiologies. When these degenerative changes are advanced, irreversible, and unresponsive to non-operative management, it may become necessary to replace all or part of the natural joint with a prosthetic device. When such a replacement becomes necessary, the prosthetic device that is implanted is often secured to the natural bone using bone cement.

Bone cement that is used to secure prosthetic devices to bone is generally comprised of a liquid monomer component that polymerizes about a polymeric powder component. In this regard, bone cement is generally formed from a methyl methacrylate monomer, polymethyl-methacrylate (PMMA), or methyl methacrylated-styrene homo- or copolymer. The polymeric powder component of bone cement can comprise particles composed of spherical beads that may be obtained by a suspension polymerization process. The beads are generally sieved to comply with particular size specifications. The powder component may also comprise particles that have been milled or crushed.

The preparation of bone cement generally involves mixing the polymer and monomer components in a suitable reaction vessel to form the bone cement. Generally it is necessary that the components of bone cement be uniformly and thoroughly mixed so that a homogenous product is obtained. Increased homogeneity of the blend and minimal porosity are particularly desirable in providing a cement mixture that is easy to work with, yet maintains satisfactory mechanical properties. In producing bone cement it is desirable to maintain the liquid and the powder components separate until just prior to use and to avoid exposure of the components to the atmosphere because of the potentially malodorous and volatile nature of the bone cement components.

Bone cements may offer an adhesive property to further couple the implant to the host bone. Cement bond strength can be a function of both true adhesion and micro-mechanical interlock that can be established between the cement and the bone opposing surface of the implant (in some examples such as grit-blasted or porous metal surfaces). Micro-mechanical interlock is influenced significantly by cement viscosity, with very high viscosity cements lacking the ability to establish a superior micro-mechanical interlock. Both pre-dough and doughy cement surfaces that have been exposed to air for a period of time can form a leathery skin via monomer liquid evaporation. These leathery surfaces can be especially poorly suited to forming a good micro-mechanical interlock and therefore, may possess virtually no adhesive properties. Application of low viscosity or medium viscosity cement directly to implants is not practical as it typically runs off of the implant. As a result, a surgeon must try to balance time, mess, and interface quality.

### SUMMARY OF THE DISCLOSURE

The current application pertains to developments in bone cement form preparation that allow a bone cement material to retain malleability and adhesiveness longer than previously available bone cements. The doughy bone cement material may be pre-shaped into a desired planar or three-dimensional form while retaining sufficient malleability and adhesiveness for a surgeon or medical practitioner to utilize. The shaped forms and structures may provide beneficial effects including but not limited to, reduced operating room time, reduced material usage, reduced operating room time and material usage, and reduced malodorous vapors within the operating room. The current application provides doughy bone cement forms, kits comprising doughy bone cement forms, over-sized doughy bone cement forms, kits comprising over-sized doughy bone cement forms, and kits for preparing doughy bone cement forms.

In an embodiment, the current application provides kits comprising a doughy bone cement form and a membrane comprising at least one removable panel. In an aspect of the kit, the membrane is flexible. In an aspect, the membrane comprises at least one component selected from a group of components comprising a thin section, perforations, and a tear starting notch. An aspect of the embodiment provides a membrane comprising silicone. In an aspect, the working time of the doughy bone cement continues at least one minute after removal of the panel at operating room temperature. In an aspect, the working time of the doughy bone cement continues at least five minutes after removal of the panel at operating room temperature. An aspect provides an operating room temperature within the range of 15° C. and 19° C. An aspect provides bone cement forms selected from the group comprising planar forms, three-dimensional forms and bone cement mantle forms useful in an arthroplastic procedure. Arthroplastic procedures may include, but are not limited to, knee, hip, elbow and shoulder arthroplastic procedures. Aspects of the claimed kits provide doughy bone cement forms selected from the group comprising planar forms and three-dimensional forms.

In accordance with one aspect of the present disclosure, a bone cement forming kit is provided and comprises a malleable bone cement material, and a flexible membrane encapsulating the malleable bone cement material, wherein the flexible membrane has at least one removable panel. In accordance with this embodiment, the flexible membrane is configured to form an aperture that exposes the malleable bone cement material once the panel is removed.

In accordance with another aspect of the present disclosure, a malleable bone cement material is provided and comprises a liquid component formed from at least one of polymethylmethacrylate, methyl methacrylate monomer, poly(methyl methacrylate), methyl methacrylate-styrene homopolymer and methyl methacrylate-styrene homopolymers; a powder component selected from homopolymers or copolymers of acrylic acid esters, methacrylic acid esters, styrene, vinyl derivatives and mixtures thereof; and a reactive liquid component containing organic monomers selected from methylmethacrylate, homolog esters of methacrylic acid and mixtures thereof. In accordance with this embodiment, the malleable bone cement material is capable of being formed into a desired shape for up to about 30 minutes after being exposed to a temperature within the range of about 15° C. to about 19° C.

Doughy bone cement forms selected from the group of bone cement mantle forms useful in an arthroplastic procedure selected from the group of arthroplastic procedures comprising knee, hip, elbow and shoulder arthroplastic procedures are provided.

A kit comprising an oversize doughy bone cement form and a membrane comprising at least one removable panel is provided. In an aspect of the kit, the working time of the oversize bone cement form continues at least one minute after removal of the panel at operating room temperature. An aspect provides that the membrane is comprised of silicone. In an aspect, the kit further comprises a sizing tool. The sizing tool may shape the doughy bone cement form.

Oversize doughy bone cement forms for trim to fit use wherein the working time of the form continues at least one minute at operating room temperature are provided.

Kits for producing a doughy bone cement form comprising a flexible pouch with a geometric seal profile similar to the desired bone cement form, wherein said pouch comprises at least one removable panel, a powder polymer component and a liquid component are provided. In an aspect of the kit, after removal of the panel, the doughy bone cement form retains the desired bone cement form and the working time of the form continues at least one minute at operating room temperature. An aspect of the kit provides a desired bone cement form is selected from the group comprising planar forms, three-dimensional forms and bone cement mantle forms useful in an arthroplastic procedure. Arthroplastic procedures may include, but are not limited to, knee, hip, elbow and shoulder arthroplastic procedures.

Kits for producing a doughy bone cement form comprising a mold with a threaded aperture, with a void similar to the desired bone cement form, a powder polymer component and a liquid component and wherein the doughy bone cement form retains the desired bone cement form upon removal from the mold and the working time of the doughy bone cement form continues at least one minute at operating room temperature upon removal from the mold are provided.

### DETAILED DESCRIPTION

The embodiments of the present application described below are not intended to be exhaustive or to limit the teachings of the present application to the precise forms disclosed in the following detailed description. Rather, the embodiments are chosen and described so that others skilled in the art may appreciate and understand the principles and practices of the present application.

Unless defined otherwise, all technical and scientific terms used herein have the same meaning as commonly understood by one of ordinary skill in the art to which this application belongs. Although any method and materials similar or equivalent to those described herein can be used in the practice or testing of the present application, the specific methods and materials are now described.

Bone cement preparation and usage are time-sensitive processes. The preparation process may result in malodorous vapors or deleterious temperature fluctuations. Once bone cement components are mixed, the bone cement mixture passes through several stages: a pre-doughy, low viscosity stage, a medium-high viscosity doughy, workable stage, and semi-solid curing stage. The pre-doughy stage is generally not workable as the cement is too runny to shape or place in position. During mixing of the bone cement components and the early pre-dough stage, the components may release noxious vapors, experience extreme temperature fluctuations or exhibit other undesirable characteristics. The surgeon or medical practitioner works most effectively with bone cement during the working time (described below herein). As the bone cement enters the curing stage, desirable properties such as malleability and adhesiveness decrease.

The kits and devices of the current application pertain to advancements in bone cement chemistry or bone cement storage that expand the working time of doughy bone cement. In some embodiments, the doughy bone cement may be formed into a doughy bone cement form with a desired bone cement form prior to the surgery. In some embodiments the doughy bone cement may be formed into a desired doughy bone shape intra-operatively. In some embodiments, the doughy bone cement may be shaped generally into a desired bone cement form prior to surgery and patient specific adjustment to the desired doughy bone cement shape may be shaped intra-operatively.

Bone cement includes but is not limited to polymethylmethacrylate (PMMA) bone cement and those formed from a methyl methacrylate monomer and poly (methyl methacrylate) or methyl methacrylate-styrene homo- or co-polymer. Such cements are generally made from mixing two components, either prior to or during the clinical procedure resulting in a compound which hardens over time. The cement components may comprise a powder polymer component comprising a polymer selected from homopolymers or copolymers of acrylic acid esters, methacrylic acid esters, styrene, vinyl derivatives and mixtures thereof. Cement components may further comprise a reactive liquid component comprising reactive organic monomers selected from methylmethacrylate, homolog esters of methacrylic acid or their mixtures. Cements among those useful herein include, but are not limited to Palacos R®, Cobalt HV®, SmartSet HV®, Simplex P®, Cobalt MV® and SmartSet MV®. Bone cement may be used in a variety of applications including but not limited to, adhering an implant to bone material, adhering bone material to bone material, filling a bone void, dental implants and forming a temporary cement spacer. See for example, U.S. Pat. No. 7,594,578 and U.S. Application 61/485,975, herein incorporated by reference in their entirety.

“Doughy” bone cement encompasses malleable bone cement to which a latex gloved finger lightly pressed into the cement does not stick. By “working time” is intended the period from on-set of the dough time to the curing time. Typically the on-set of dough time begins approximately 3-3:30 minutes after the powder and liquid are combined. It is recognized that temperature impacts the duration of the working time. Prior to the current application, the curing time was reached around 8 minutes (at room temperature) and around 11-12 minutes at operating room temperature. The present kits provide a doughy bone cement with a working time that continues from at least about 1 minute to about 30 minutes at operating room temperature after removal of at least one panel from a flexible pouch, membrane or mold encapsulating the doughy bone cement. The working time continues, lasts, persists, extends, occurs for, abides, holds, or remains until the curing time begins.

A “form” is an object with shape and structure, the shape and structure of which may be alterable. It is envisioned that a doughy bone cement form will be shaped, guided, altered, manipulated, placed, pressed, pushed, bent, folded, turned, squeezed, compressed, wrapped, tapered, layered, stretched, pinched or pulled into the final desired form and position prior to curing. Forms of particular interest may include, but are not limited to, planar forms, three-dimensional forms and bone cement mantle forms. A “planar” form is generally flat. The thickness or depth of a planar form may range from about 0.5 mm to about 20 mm, from about 1 mm to about 15 mm, from about 1.5 mm to about 10 mm, from about 2 mm to about 10 mm, from about 3 mm to about 10 mm, from about 4 mm to about 7 mm. It is recognized that during use a planar form may be shaped, guided, altered, manipulated, placed, pressed, pushed, bent, folded, turned, wrapped, tapered, layered, stretched, pinched or pulled into a three-dimensional form. It is recognized that during use a planar form may be compressed unevenly resulting in different thicknesses.

A “three-dimensional” form generally occurs in three dimensions including both regular and irregular shapes. A bone cement “mantle form” is a shape or structure useful in an arthroplastic procedure. It is recognized that a mantle form useful in a first arthroplastic procedure may or may not be useful in a second arthroplastic procedure. Mantle forms may include but are not limited to, planar forms, discs, pads, sheets, blankets, three-dimensional forms, and geometries corresponding to the bone opposing surface of a prosthetic implant, a bone surface receiving geometry, caps, cups, and bowls.

Doughy bone cement forms may be utilized in various medical applications including, but not limited to, an arthroplastic procedure, an orthopedic implant, a dental implant, a bone fixation device, a scaffold, or a custom made or generic form for repairing a bone defect caused by surgical intervention or disease. As referenced herein, the term “implant” may refer to an entire implant as a whole, or a portion thereof; portions may be as large or as small as necessary to accomplish the desired effect. “Arthroplastic procedure” encompasses any procedure to improve the integrity or function of a joint including, but not limited to, full or partial joint replacement of the hip, knee, elbow, shoulder, ankle, wrist, medical implant, orthopedic implant, including but not limited to an acetabular cup, a knee implant, a shoulder implant, a femoral implant or femoral resurfacing system, and interpositional reconstruction.

A “desired” bone cement form has a shape or structure that resembles, is similar to, or matches a geometrical configuration of use in a particular medical application. It is recognized that a desired bone cement form for one medical application may or may not be a desired bone cement form for a different medical application. By way of example, and without limitation, a desired bone cement form for a hip replacement may differ from a desired bone cement form for a knee replacement. Factors that may affect the desirability of a particular bone cement form may include, but are not limited to, the medical application, the type of surgery, the surgery location, the subject's size, the subject's lifestyle, the subject's age, and the subject's gender.

Operating room temperatures are typically lower than room temperature. The operating room temperature may range from about 0° C. to about 23° C., from about 4° C. to about 22° C., from about 8° C. to about 20° C., from about 12° C. to about 20° C., from about 14° C. to about 19° C., from about 15° C. to about 19° C. and from about 16° C. to about 18° C. Temperature ranges above or below the indicated operating room temperature may alter the duration of the working time and the curing time onset.

Kits comprising a membrane comprising at least one removable panel are provided. Membranes suitable for use in a kit may include, but are not limited to, flexible, semi-flexible, and rigid materials suitable for medical or veterinary uses such as, but not limited to, silicone. A membrane may be removably disposed on the inner surface of a mold cavity. The membrane may include features to allow for easy separation from the doughy bone cement form. Such features may include, but are not limited to, a removable panel, a thin section, fine perforations, and a tear starting notch or cut. The membrane may have a low tear strength such as some silicone formulations. A membrane or a mold of the current application may include components such as, but not limited to, a vacuum port, a threaded aperture, and an orthopedic implant.

Membranes, flexible pouches and molds of the current application may comprise a removable panel. A panel is a distinct part of a membrane, flexible pouch or mold; a panel may be delineated by markings, seams, perforations, low tear strength regions, thin sections or lines. By “removable panel” is intended that removal of the panel may be accomplished without significant alteration to the shape of the bone cement form and that the exposed bone cement form may retain its shape for a period of time. It is recognized that removal of the panel may be accomplished by cutting, peeling, slicing, or tearing the panel. Without being limited by mechanism it is envisioned that removal of the panel may create an aperture through which the doughy bone cement form may exit the remainder of the membrane while retaining the desired bone cement form or that removal of the panel may facilitate removal of the remainder of the membrane from the dough bone cement form. It is further envisioned that removal of the panel may create an aperture through which an implant, an implant component, a bone, or a bone structure may be inserted into, positioned within, placed in, affixed to, or attached to the doughy bone cement form. Placement of an implant, implant component, a bone or bone structure within the doughy bone cement form may occur prior to complete removal of the membrane from the doughy bone cement form, upon complete removal of the membrane, or after complete removal of the membrane from the dough bone cement form. It is recognized that the membrane may modulate the morphology of the doughy bone cement form.

Perforations of a membrane in the instant application encompass a series of holes or weaker material that serve as an aid in separation. A “thin section” of a membrane is a portion of the membrane having less thickness than another portion of the membrane. A “tear starting notch” is a small slit such as a cut or incision positioned in a manner to facilitate a tear, rip, opening, separation, or divide. Components of the membrane may be comprised of the same, different, or additional materials as the remainder of the membrane.

Kits comprising an oversize doughy bone cement form and a membrane comprising at least one removable panel are provided. Oversize doughy bone cement forms may be planar forms, three-dimensional forms, and bone cement mantle forms. The oversize forms comprise more doughy bone cement than will remain within the recipient subject. Oversize doughy bone cement forms may be used in a trim to fit capacity. A trim to fit capacity supplies a predetermined amount of material that the practitioner can reduce to yield the desired final amount and shape. The excess doughy bone cement provides more than necessary doughy bone cement for the medical practitioner, allowing the medical practitioner to manipulate, shape, position, or mold the doughy bone cement prior to removal of excess doughy bone cement. Oversize may refer to the entire doughy bone cement form or one or more dimensions, shapes, or portions of the doughy bone cement form. If one or more dimensions, shapes or portions of the doughy bone cement form are oversize, one or more other dimensions, shapes or portions of the form may not be oversize. Without being limited by example, an oversized cap shaped doughy bone cement form may provide the desired diameter while it may be desirable to reduce the cap wall length.

Kits may further comprise a sizing tool. A sizing tool is a device that facilitates removal of excess material. In an embodiment a sizing tool may also shape the material. Sizing tools may vary for different medical applications. Sizing tools may include, but are not limited to, scalpels, blades, knives, wires, lasers and scissors.

Kits for preparing a doughy bone cement form comprising a flexible pouch with a geometric seal profile similar to a desired bone cement form wherein the pouch comprises at least one removable panel, a powder polymer component and a liquid component are provided. Materials suitable for the flexible pouches of the instant application are known in the art and include, but are not limited to, materials described in U.S. Pat. No. 7,594,578 and U.S. Patent Application 61/485,975, the disclosures of which are herein incorporated by reference in their entireties.

In an embodiment, the flexible pouch comprises a first chamber. The geometric seal profile of the first chamber of the flexible pouch is similar to a desired bone cement form. “Geometric seal profile” is intended to encompass the pattern, template, lay-out, arrangement, design, shape, or configuration of a seal of the first chamber. The geometric seal profile encompasses the circumferential seal or seals of the first chamber as well as any interior or semi interior seals. The geometric seal profile is a predetermined pattern or template that results in the pouch flexing into the configuration of a desired bone cement form when bone cement is within the first chamber of the pouch. Other configuration factors including, but not limited to, regions of variable wall thickness in the pouch, regions of altered rigidity, and regions of altered stretching capacity, may contribute to preparation of a desired bone cement form. In some embodiments, the geometric seal profile may decrease the flexibility of one or more regions of the flexible pouch. The first chamber may store the powder polymer component of bone cement prior to the introduction of the liquid component of bone cement. In an embodiment, the first chamber comprises at least one removable panel.

The liquid component is mixed with the powder polymer component in the first chamber to prepare the doughy bone cement. In various embodiments, the flexible pouch comprises a second chamber. The second chamber may store the liquid component of the bone cement prior to mixing the bone cement components. It is envisioned that an incubation period may occur after combining the bone cement components. The incubation period may extend at least until the bone cement enters the working time. After removal of the removable panel from the pouch, the doughy bone cement form retains the desired bone cement form and the working time of the doughy bone cement continues for at least one minute after removal of the removable panel. After removal of the removable panel, the working time may continue for at least the durations discussed elsewhere herein.

Kits for preparing a doughy bone cement form comprising a mold with a threaded aperture and a void similar to the desired bone cement form, a powder polymer component, and a liquid component are provided. Mold is intended to encompass a semi-rigid or rigid structure with a void in a predetermined configuration, particularly a desired bone cement form. The void may be lined with a flexible membrane to facilitate separation of the doughy bone cement and the mold. After removal of the removable panel, the working time may continue for at least the durations discussed elsewhere herein. Threaded aperture is intended to encompass an aperture with threads located on the interior of the aperture or on a neck surrounding the aperture. A threaded aperture may facilitate access to the mold for one or more purposes including but not limited to, delivery of a bone cement component, removal of air, air bubbles or air pockets, release of vapors, and release of pressure. A threaded aperture allows attachment of threaded devices as desired by the practitioner.

It is recognized that the void will be of a size and shape that will determine the shape and dimensions of the desired bone cement form. In various embodiments, the shape of the void and the resulting doughy bone cement form will substantially conform to the profile of an implant. It is recognized that the dimensions of the void and the resulting desired bone cement form may vary along the surface of an implant. The void and resulting doughy bone cement form may range from about 1 mm to about 15 mm, from about 2 mm to about 10 mm, or from about 3 mm to about 7 mm in depth.

All publications, patents, and patent applications mentioned in the specification are indicative of the level of those skilled in the art to which this invention pertains. All publications, patents, and patent applications are herein incorporated by reference to the same extent as if each individual publication or patent application was specifically and individually incorporated by reference.

While an exemplary embodiment incorporating the principles of the present application has been disclosed hereinabove, the present application is not limited to the disclosed embodiments. Instead, this application is intended to cover any variations, uses, or adaptations of the application using its general principles. Further, this application is intended to cover such departures from the present disclosure as come within known or customary practice in the art to which this present application pertains and which fall within the limits of the appended claims.

The terminology used herein is for the purpose of describing particular illustrative embodiments only and is not intended to be limiting. As used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath”, “below”, “lower”, “above”, “upper” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the FIGURES. For example, if the device in the FIGURES is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations).

What is claimed is:

1. A bone cement forming kit, comprising:
a malleable bone cement material; and
a flexible membrane encapsulating the malleable bone cement material, the flexible membrane having at least one removable panel;
wherein the flexible membrane is configured to form an aperture that exposes the malleable bone cement material once the panel is removed.

2. The bone cement forming kit of claim 1, further comprising a sizing tool selected from a scalpel, a blade, a knife, a wire, a laser and scissors.

3. The bone cement forming kit of claim 1, wherein the malleable bone cement material is formed from at least one of polymethylmethacrylate, methyl methacrylate monomer, poly(methyl methacrylate), methyl methacrylate-styrene homopolymer and methyl methacrylate-styrene homopolymer.

4. The bone cement forming kit of claim 1, wherein the malleable bone cement material further comprises a powder component selected from homopolymers or copolymers of acrylic acid esters, methacrylic acid esters, styrene, vinyl derivatives and mixtures thereof.

5. The bone cement forming kit of claim 1, wherein the malleable bone cement material further comprises a reactive liquid component containing reactive organic monomers selected from methylmethacrylate, homolog esters of methacrylic acid and mixtures thereof.

6. The bone cement forming kit of claim 1, wherein the flexible membrane is a silicone membrane and includes at least one of a perforated section or a notched section.

7. The bone cement forming kit of claim 1, wherein the malleable bone cement material is capable of being formed into a desired shape for up to about 30 minutes after being removed from the flexible membrane and exposed to a temperature within the range of about 15° C. to about 19° C.

8. The bone cement forming kit of claim 1, wherein the malleable bone cement material is pre-shaped into a planar, three-dimensional or mantle form.

9. The bone cement forming kit of claim 1, wherein the flexible membrane is removably disposed on a mold cavity.

10. The bone cement forming kit of claim 9, further comprising a vacuum port, a threaded aperture or an orthopedic implant associated with the flexible membrane.

11. The bone cement forming kit of claim 1, wherein the malleable bone cement material is formable into a bone cement mantle for an arthroplasty procedure.

12. The bone cement forming kit of claim 11, wherein the bone cement mantle is formable into a shape selected from the group including planar forms, discs, pads, sheets, blankets, three-dimensional forms, and geometric forms that correspond to a bone opposing surface of a prosthetic implant, a bone surface receiving geometry, caps, cups or bowls.

13. The bone cement forming kit of claim 11, wherein the arthroplasty procedure is selected from a hip procedure, an elbow procedure, and a shoulder procedure.

14. A bone cement forming kit of claim 1, wherein the flexible membrane comprises a flexible pouch having a first chamber, a powder polymer component and a liquid component, wherein the first chamber of the flexible pouch has a geometric seal profile similar to a desired bone cement form.

15. The bone cement forming kit of claim 14, wherein said flexible pouch further comprises a second chamber.

16. A malleable bone cement material, comprising
a liquid component formed from at least one of polymethylmethacrylate, methyl methacrylate monomer, poly(methyl methacrylate), methyl methacrylate-styrene homopolymer and methyl methacrylate-styrene homopolymers;
a powder component selected from homopolymers or copolymers of acrylic acid esters, methacrylic acid esters, styrene, vinyl derivatives and mixtures thereof; and
a reactive liquid component containing organic monomers selected from methylmethacrylate, homolog esters of methacrylic acid and mixtures thereof;
wherein the malleable bone cement material is capable of being formed into a desired shape for up to about 30 minutes after being exposed to a temperature within the range of about 15° C. to about 19° C.

17. The malleable bone cement material of claim 16, wherein the material is pre-shaped into a planar, three-dimensional or mantle form.

18. The malleable bone cement material of claim 17, wherein the mantle form is selected from the group including planar forms, discs, pads, sheets, blankets, three-dimensional forms, and geometric forms that correspond to a bone opposing surface of a prosthetic implant, a bone surface receiving geometry, caps, cups or bowls.

19. The malleable bone cement material of claim 16, wherein the material is formable into a bone cement mantle for an arthroplasty procedure

20. The malleable bone cement material of claim 19, wherein the arthroplasty procedure is selected from a hip procedure, an elbow procedure, and a shoulder procedure.
